(19) United States
(12) Patent Application Publication
(10) Pub. No.: US 2021/0177816 A1
(43) Pub. Date: Jun. 17, 2021

(54) nAChR-alpha7 Agonists and nAChR-alpha7 Antagonists for Treating Ulcerative Colitis (UC) and Crohn's Disease (CD)

(71) Applicants: The Johns Hopkins University, Baltimore, MD (US); Oklahoma Medical Research Foundation, Oklahoma City, OK (US)

(72) Inventors: Xuhang Li, Clarksville, MD (US); Philip Alex, Bel Air, MD (US); Michael B. Centola, Oklahoma City, OK (US)

(21) Appl. No.: 17/080,360

(22) Filed: Oct. 26, 2020

Related U.S. Application Data
(63) Continuation of application No. 13/805,811, filed on Mar. 5, 2013, now abandoned.
(60) Provisional application No. 61/358,481, filed on Jun. 25, 2010.

(51) Int. Cl. A61K 31/444; A61K 31/439; A61K 31/422; A61K 35/58; A61K 31/4748; A61K 31/46; A61K 38/17; A61K 31/5513

(52) U.S. Cl. CPC A61K 31/444; A61K 31/439; A61K 31/422; A61K 35/58; A61K 31/4748; G01N 2333/70571; A61K 38/17; A61K 31/5513; G01N 2800/065; G01N 2500/10; A61K 31/46

(57) ABSTRACT

Agonists and antagonists of nAChRα7 and their use as therapeutic agents for treating and managing inflammatory bowel diseases (IBD), such as Crohn's disease (CD) and ulcerative colitis (UC), are disclosed.

### CROSS-REFERENCE TO RELATED APPLICATION

This application is a continuation of U.S. patent application Ser. No. 13/805,811, filed Mar. 5, 2013, which is a § 371 U.S. National Entry Application of PCT/US2011/041730, filed Jun. 24, 2011, which claims the benefit of U.S. Provisional Application No. 61/358,481, filed Jun. 25, 2010, each of which is incorporated herein by reference in its entirety.

### BACKGROUND

Inflammatory bowel diseases (IBD), including, but not limited to Crohn's disease (CD) and ulcerative colitis (UC), are chronic intestinal disorders that affect approximately 3.4 million people in Western countries alone and result in enormous suffering and health-care costs. For the past several decades, scientists and clinicians have been puzzled by the observation that smoking tobacco products has a beneficial influence on the course of UC and a detrimental effect on the course of CD. The effect of smoking and the role of nicotine on the course of UC and CD, however, are not well understood.

### SUMMARY

In some aspects, the presently disclosed subject matter provides a method for treating an inflammatory bowel disease (IBD) in a subject in need of treatment thereof, the method comprising administering a nicotinic acetylcholine receptor alpha 7 (nAChRα7) antagonist to the subject in an amount effective to modulate an activity of nAChRα7 in at least one cell of the subject, whereby the modulating of the activity of nAChRα7 in the at least one cell treats the IBD in the subject. In particular aspects, the IBD comprises Crohn's disease (CD).

In other aspects, the presently disclosed subject matter provides a method for treating an inflammatory bowel disease (IBD) in a subject in need of treatment thereof, the method comprising administering an nAChRα7 agonist to the subject in an amount effective to modulate an activity of nAChRα7 in at least one cell of the subject, whereby the modulating of the activity of nAChRα7 in the at least one cell treats the IBD in the subject. In particular aspects, the IBD comprises ulcerative colitis (UC).

In further aspects, the presently disclosed subject matter provides a method for modulating an activity of nAChRα7 in at least one immune cell type, the method comprising contacting the at least one immune cell type with an nAChRα7 antagonist or an nAChRα7 agonist in an amount effective to modulate the activity of nAChRα7 in the at least one immune cell type.

In other aspects, the presently disclosed subject matter provides a method for reducing the risk of developing colorectal cancer in a subject having a chronic gastrointestinal tract inflammation, the method comprising administering an nAChRα7 antagonist or an nAChRα7 agonist to the subject in an amount effective to modulate an activity of nAChRα7 in one or more immune cells of the gastrointestinal tract, whereby modulating the activity of nAChRα7 alters an inflammatory response in the one or more immune cells of the gastrointestinal tract, thereby reducing the risk of developing colorectal cancer.

In some aspects, the presently disclosed subject matter provides a method for identifying a compound or agent that modulates an activity of nAChRα7 in at least one cell expressing nAChRα7, the method comprising: (i) contacting the at least one cell expressing nAChRα7 with a candidate compound or agent; (ii) determining the activity of nAChRα7 in the at least one cell expressing nAChRα7 that has been contacted with the candidate compound or agent; (iii) determining the activity of nAChRα7 in at least one control cell expressing nAChRα7 that has not been contacted with the candidate compound or agent; and (iv) comparing the activity of nAChRα7 in the at least one cell that has been contacted with the candidate compound or agent to the activity of nAChRα7 in the at least one control cell; wherein a difference in the activity of nAChRα7 in the at least one cell that has been contacted with the candidate compound or agent and the at least one control cell identifies a candidate compound or agent that modulates the activity of nAChRα7 in at least one cell.

In yet other aspects, the presently disclosed subject matter provides a method for predicting a therapeutic effect of administering a modulator of nAChRα7 expression to a subject afflicted with IBD resulting from an abnormal level of gene or protein expression of nAChRα7, wherein the modulator of nAChRα7 expression is an nAChRα7 antagonist or an nAChRα7 agonist, the method comprising: (a) measuring a level of gene or protein expression of nAChRα7 in a tissue or cell of the subject before administering the nAChRα7 modulator; (b) administering an nAChRα7 modulator to the subject in an amount effective to alter an nAChRα7 gene or protein expression level in a tissue or cell of the subject; (c) measuring a level of gene or protein expression of nAChRα7 in a tissue or cell from the subject after administering the nAChRα7 modulator; and (d) determining an alteration in the level of gene or protein expression of nAChRα7 in the tissue or cell after administering the nAChRα7 modulator from the level of gene or protein expression in the tissue or cell before administering the nAChRα7 modulator; wherein an alteration in the level of nAChRα7 gene or protein expression in the tissue or cell predicts a therapeutic effect of the modulator of nAChRα7 expression to a subject afflicted with IBD.

In some aspects, the presently disclosed subject matter provides a pharmaceutical composition comprising a specific agonist or a specific antagonist of a nicotinic acetylcholine receptor alpha 7 (nAChRα7) in an amount effective to modulate the function of nAChRα7 to treat or prevent an inflammatory bowel disease (IBD) in a subject in need of treatment thereof.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows representative hierarchical clustering from microarray profiles showing distinct expression patterns in IBD. Each row represents a gene and each column represents an independent biological sample. Clustering analysis of gene expression profiles from immune cells showed differential patterns that clustered into four groups as shown: normal (N), CD, UC, and normal (N). The shading indicates either downregulated or over expressed, whereas black indicates similarly expressed;

FIG. 2 shows the discriminatory potential of the 10 genes identified by DFA. DFA was used to select maximal discriminatory variables and UC, CD, and healthy controls were grouped into three distinct positions;

FIG. 3 shows the differential gene expression pattern of nAChRα7 in UC, CD, and unaffected controls identified by gene microarrays and confirmed by QRT-PCR. Left panel: Microarray data: normalized expression levels of nAChRα7 from immune cells depict a unique differential gene expression pattern in UC and CD. N: neutrophils; P: PMBCs; Ctrl: controls. Right panel: validity of microarray results by QRT-PCR. QRT-PCR confirmed gene expression results of nAChRα7 and five (5) other selected genes (data not shown) from PBMCs;

FIG. 4 demonstrates that nAChRα7 mediates distinct immunomodulatory profiles in lymphocytes from CD vs. UC. Ratios of the cytokine profiles from supernatants of treated transformed lymphocytes relative to untreated cells in UC and CD are represented (one of at least three similar experiments). Treatments included nicotine [2 μM (+) and 20 μM (++)] and pretreatment with selective antagonist α-bungarotoxin [2 nM (+) and 20 nM (++)] before addition of nicotine. Panel A: cytokines IL-10 and cytokine/chemokine IL-8. Panel B: chemokines. For all unaffected controls, addition of nicotine or α-bungarotoxin by itself depicted insignificant cytokine/chemokine changes (not shown). Nic: nicotine. Antg: selective antagonist α-bungarotoxin;

FIG. 5 shows opposite effects of nicotine and an nAChRα7-specific agonist on the course of “CD-like” TNBS-colitis and “UC-like” DSS-colitis. These IBD mouse models were considered “CD-like” or “UC-like” because not only do their disease phenotype resemble either human CD or UC, respectively, but also their cytokine profiles are similar to human CD or UC, particularly the chronic models of TNBS- or DSS models, as demonstrated previously (Alex et al, 2009 IBD, 15(3), 341-352). These experimental mouse colitis models were generated as described previously (Alex et al, 2009 IBD, 15(3), 341-352). Ethanol: control for TNBS; Nic: nicotine; a7 Ag: nAChRα7 agonist PNU 282987 (PNU);

FIG. 6 shows that nAChRα7 agonist is not only preventative, but also therapeutic for UC-like DSS-colitis. In an acute model, mice were treated with or without nAChRα7 agonist drug for five days. DSS were then given for seven days. Clinical activity scores were assessed. In a chronic model, mice were given DSS for seven days followed by seven days water. These DSS-water cycles were repeated three more times continuously. At the end of the fourth cycle, mice develop chronic colitis, with a clinical activity score of approximately three, even without DSS. At this point, nAChRα7 agonist was given in one set of mice and all mice were followed for eleven days. In both acute model and chronic model, disease (colitis) essentially disappeared and the mice appeared indistinguishable from control mice (no DSS treatment). The arrow indicates the start (day 53) of nAChRα7 agonist treatment in the chronic model;

FIG. 7 shows that pre-treatment of drug MLA, an nAChRα7-specific antagonist, can effectively prevent mice from developing CD-like TNBS colitis. C57BL/6. Mice were pretreated either with PBS (control), or nicotine, PNU (α7 agonist), or MLA drug by peritoneal injection once daily for five days. Mice were then induced to develop colitis for seven days with either TNBS (in 50% ethanol) or 50% ethanol alone as a control. Disease activity of all mice was monitored daily. Disease (colitis) essentially disappeared in the TNBS-treated mice that were pretreated with MLA (α7 antagonist+TNBS). These MLA-treated mice appeared indistinguishable from no TNBS control mice (ethanol). In contrast, mice pretreated with nicotine (nicotine+TNBS), or PNU (α7 agonist+TNBS) showed either no therapeutic benefit, even worsening disease activity, compared to TNBS-treatment alone (TNBS);

FIG. 8 shows that treatment with a nAChRα7-specific antagonist (Antg; MLA) effectively reverses the disease course of chronic CD-like TNBS-induced colitis. Induction of chronic TNBS-colitis model was described previously (Alex et al, 2009 IBD, 15(3), 341-352). The DAI of antagonist-treated colitis mice is similar to control mice [treated with ethanol (E-OH)]. nAChRα7 agonist (Ag; PNU) and nicotine exhibited no significant effect. Statistical analysis were done by ANOVA and Tukey's test; p<0.05. N≥12 per group;

FIG. 9 shows that treatment with nAChRα7 antagonist reverses TNBS-induced colonic inflammation: (A) the colon from mice with chronic TNBS-induced colitis treated is shorter, inflamed, and filled with bloody and soft stool; (A-c), while the colon from antagonist-treated colitis mice is a completely normal-looking (A-d), almost indistinguishable from the control mice (A-a &b); (B) Histological analysis by H&E staining shows that antagonist-treated TNBS-mice have normal colonic mucosa with little sign of inflammation (B-c), as seen in untreated TNBS-mice (B-b). Representatives of at least ten independent experiments are shown;

FIG. 10 shows that neither nAChRα7-specific agonist nor antagonist has any therapeutic effects on DSS- or TNBS-induced colitis of nAChRα7 deficient mice, demonstrating the specificity of the drugs. Wild type (WT) nAChRα7 deficient (nAChRα7 knockout; α7KO) mice, both in C57B/6 background, were induced to develop colitis, either with DSS (A) or TNBS (B). While agonist (PNU) and antagonist (MLA) effectively prevent DSS- and TNBS-induced colitis, respectively, in WT mice, they exhibit no effect at all on either type of colitis in nAChRα7 KO mice. These data further demonstrate the target specificity of these agonists and antagonists to nAChRα7; Statistical differences in measured values were assessed by ANOVA and Tukey's test at the 5% level of significance. N≥6 per group;

FIG. 11 shows that mice that underwent vagotomy completely lost the protective effect of nAChRα7 agonist on the development of colitis: (A) Before and after vagotomy: Vagotomy was performed based on a protocol modified from previous reports (Verma-Gandhu et al, Gut 2007; 56:358-364). Briefly, left vagal branch (approximately 5-10 mm; indicated by a double-headed arrow) at the gastroesophageal junction (marked by white arrows) of C57B/6 mice (7 weeks old) was cut with an electrocuttery under microscope magnification (10×) (S, stomach; E, esophagus). Surgical opening was closed with absorbable sutures. Sham surgery involved the same procedure without excision of the vagus nerve (just exposure). Vagotomized mice were given 7 days to recover. 100% of all 30 mice that underwent surgery (vagotomy and sham) survived with no complications and recovered to normal. Mice were then administered nAChRα7 agonist by IP for 5 days before DSS treatment (7 days) to induce colitis; (B) Vagotomized mice (Vagot) did not exhibit the protective effect of nAChRα7 agonist on colitis at all while mice with sham surgery (Sham) responded as nicely as those without any surgery (see FIGS. 5 and 6). Statistical differences in measured values were assessed by a Wilcoxon rank-sums test (P<0.05). N≥5 per group;

FIG. 12 shows that nAChRα7 agonist is therapeutic for GPX 1/2 DKO-colitis. Mice deficient in two glutathione peroxidases (GPX), Gpx1 and Gpx2, [Gpx1/2-double knockout (DKO) mice] were prone to ileocolitis on a mixed C57BL/6 and 129S1/SvJ (B6.129) genetic background. Clinical activity scores were assessed. While controls (from mixed C57BL/6 and 129S1/SvJ (B6.129) genetic background) do not develop severe colitis, GPX 1/2 DKO mice were found to develop severe colitis with a clinical activity score of approximately 9. In GPX 1/2 DKO mice that were treated with nAChRα7 agonist, the development of disease (colitis) was significantly decreased. GPX ½ DKO mice treated with nAChRα7 antagonist or saline, however, did not demonstrate significant changes in colitis scores;

FIG. 13 shows that Discriminant Functional Analysis (DFA) identifies the variables of cytokines/chemokines that can sufficiently distinguish one disease type from the other and therapeutic responders from controls. DSS-induced and TNBS-induced colitis formed distinct groups that mapped away from the controls and away from each other. On the other hand, both DSS-induced mice treated with the agonist and TNBS-induced mice treated with the antagonist mapped very close to controls and each other—a clear shift of cytokine profiles from disease states toward normal state after effective treatments of respective drugs; and

FIG. 14 shows that forced swim tests show no measureable effect of nAChRα7 agonist (PNU) on mouse behavior. Tests were carried out on mice forced to swim in a cylindrical container for six minutes. Mice were divided into four groups: (a) control; (b) mice receiving IP agonist; (c) mice induced with DSS; and (d) mice receiving IP agonist and induced with DSS (n=6 in each group). The total duration of immobility in a container with 13 cm of water at 25° C. was scored. Immobility was defined when the mouse stopped struggling and remained floating motionless in the water, making only those movements necessary to keep its head above water. Day 0 and Day 7 indicate the days of DSS treatment.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. nAChRα7 Agonists and nAChRα7 Antagonists for Treating Ulcerative Colitis (UC) and Crohn's Disease (CD)

The presently disclosed subject matter, in some embodiments, has identified nicotinic acetylcholine receptor alpha 7 (nAChRα7) as a master regulator in modulating the opposite effects of smoking (nicotine) on CD vs. UC. Accordingly, the presently disclosed subject matter identifies nAChRα7 as a novel therapeutic target for IBD. The presently disclosed subject matter provides specific agonists and antagonists of nAChRα7 and their use as therapeutic agents for treating and managing UC and CD.

A. Role of Smoking (Nicotine) in IBD

Inflammatory bowel disease (IBD) is a prevalent chronic, progressive multifactorial inflammatory disorder of the gastrointestinal (GI) tract that presents in two distinct common entities: ulcerative colitis (UC) and Crohn's disease (CD). Xavier R. J., Podolsky D. K, “Unravelling the pathogenesis of inflammatory bowel disease,” Nature 448:427-434 (2007). As used herein, the phrase “inflammatory bowel disease” or “IBD” is intended to encompass two chronic diseases affecting the gastrointestinal tract known as Crohn's disease (CD) and ulcerative colitis (UC). The meaning of the terms “Crohn's disease” and “ulcerative colitis” are those definitions that are generally accepted by those of skill in the art in the medical community.

With more than a million patients diagnosed in the United States alone, IBD has a prevalence of approximately 0.2% of the Western population and has enormous suffering and health-care costs. Loftus E. V., Jr., “Clinical epidemiology of inflammatory bowel disease: Incidence, prevalence, and environmental influences,” Gastroenterology 126:1504-1517 (2004). A major complication of colonic IBD is the increased risk of developing colorectal cancer (CRC), due primarily to chronic GI inflammation. Shanahan F., “Review article: colitis-associated cancer—time for new strategies 1,” Aliment Pharmacol Ther 18 Suppl 2:6-9 (2003); Campbell B. J., Yu L. G., Rhodes J. M., “Altered glycosylation in inflammatory bowel disease: a possible role in cancer development,”Glycoconj J 18:851-858 (2001). Intriguingly, smoking has a beneficial influence on the course of UC and a detrimental effect on the course of CD. Birrenbach T., Bocker U., “Inflammatory bowel disease and smoking: a review of epidemiology, pathophysiology, and therapeutic implications,” Inflamm Bowel Dis 10:848-859 (2004); Rubin D. T., Hanauer S. B., “Smoking and inflammatory bowel disease,” Eur J Gastroenterol Hepatol 12:855-862 (2000); Thomas G. A., Rhodes J., Ingram J. R., “Mechanisms of disease: nicotine—a review of its actions in the context of gastrointestinal disease,” Nat Clin Pract Gastroenterol Hepatol 2:536-544 (2005); and Ingram J. R., Rhodes J., Evans B. K., Thomas G. A., “Preliminary observations of oral nicotine therapy for inflammatory bowel disease: an open-label phase I-II study of tolerance,” Inflamm Bowel Dis 11:1092-1096 (2005).

Ulcerative colitis (UC) has been identified as largely a disease of non-smokers and/or former smokers, and, while the initiation of smoking protects and improves the course of UC, its cessation aggravates its course. While nicotine, the active moiety in smoking, as a therapy in clinical trials demonstrates significant clinical efficacy in UC, its utility, however, is limited by considerable variation in tolerance and adverse effects. On the contrary, about 72% of CD patients are smokers, and, while smoking increases the risk of CD and worsens its course to more severe forms (structuring or fistulizing), its cessation significantly improves CD. Birrenbach T., Bocker U., “Inflammatory bowel disease and smoking: a review of epidemiology, pathophysiology, and therapeutic implications,” Inflamm Bowel Dis 10:848-859 (2004). The molecular mechanism of the opposite effects of smoking on CD and UC has remained a mystery for several decades.

B. Nicotinic Acetylcholine Receptors (nAChRs)

Nicotine has been demonstrated to trigger an inflammatory cascade through its activities on nAChRs, a family of ligand-gated ion channels formed by various homo- or heteropentameric receptor subunits, which consist of two subfamilies of 9 a (α2-α10) and 3 β (β2-β4) and are expressed in both nervous system and non-neuronal tissues (such as, immune cells and intestinal epithelial cells). Gaimarri A., Moretti M., Riganti L., Zanardi A., Clementi F., Gotti C., “Regulation of neuronal nicotinic receptor traffic and expression,” Brain Res Rev 55:134-143 (2007). Nicotine is the principal pharmacological ingredient responsible for the opposite effects on UC vs. CD. Thomas G. A., Rhodes J., Ingram J. R., “Mechanisms of disease: nicotine—a review of its actions in the context of gastrointestinal disease,” Nat Clin Pract Gastroenterol Hepatol 2:536-544 (2005).

The clinical efficacy of nicotine in UC therapy suggests that NAChRs are most likely the target for nicotine-based therapy. Its clinical application, however, is limited by the considerable variation in tolerance and adverse effects, most likely due to ligand specificity of nicotine to nAChRs. Recent studies have demonstrated that nicotine acts as an anti-inflammatory agent particularly through the inhibition of the production of pro-inflammatory cytokines from macrophages. Gallowitsch-Puerta M., Tracey K. J., “Immunologic role of the cholinergic anti-inflammatory pathway and the nicotinic acetylcholine alpha 7 receptor,” Ann N Y Acad Sci 1062:209-219 (2005). This phenomenon was defined as the “cholinergic anti-inflammatory pathway,” where neuropeptides serve as the molecular basis of a neuroimmune axis, which, through the autonomic nervous system (ANS), provides anti-inflammatory feedback to the immune system. Tracey K. J., “The inflammatory reflex,” Nature 420:853-859 (2002). This pathway is known to be mediated from neural cholinergic anti-inflammatory signals through the efferent vagus nerve and is modulated specifically by nAChRs; particularly nAChRα7 expressed on immune cells. Wang H., Yu M., Ochani M., Amella C. A., Tanovic M., Susarla S., Li J. H., Wang H., Yang H., Ulloa L., Al-Abed Y., Czura C. J., Tracey K. J., “Nicotinic acetylcholine receptor alpha7 subunit is an essential regulator of inflammation,” Nature 421:384-388 (2003); de Jonge W. J., van der Zanden E. P., The F. O., Bijlsma M. F., van Westerloo D. J., Bennink R. J., Berthoud H. R., Uematsu S., Akira S., van den Wijngaard R. M., Boeckxstaens G. E., “Stimulation of the vagus nerve attenuates macrophage activation by activating the Jak2-STAT3 signaling pathway,” Nat Immunol 6:844-851 (2005); Floto R. A., Smith K. G., “The vagus nerve, macrophages, and nicotine,” Lancet 361:1069-1070 (2003); Ghia J. E., Blennerhassett P., Kumar-Ondiveeran H., Verdu E. F., Collins S. M., “The vagus nerve: a tonic inhibitory influence associated with inflammatory bowel disease in a murine model,” Gastroenterology 131:1122-1130 (2006).

nAChRα7 has been the most intensively studied nAChR in recent years. Mazurov A., Hauser T., Miller C. H., “Selective alpha7 nicotinic acetylcholine receptor ligands,” Curr Med Chem 13:1567-1584 (2006). Interests in nAChRα7 ligands and their uses have recently increased and many of these agents are in clinical trials (but not for IBD). These advances provide molecular bases for identifying and designing/synthesizing nAChRα7-specific drugs.

C. Representative Embodiments

The presently disclosed subject matter identifies nAChRα7 as a master regulator in modulating the opposite effects of smoking (i.e., nicotine exposure) on CD vs. UC. First, it was found by gene-expression profiling of lymphocytes and neutrophils from patients with IBD that nicotinic acetylcholine receptor α7 isoform (nAChRα7) is significantly over-expressed in patients having Crohn's disease (CD), but down-regulated in patients having ulcerative colitis (UC), relative to unaffected controls. Without wishing to be bound to any one particular theory, it is hypothesized that smoking might cause opposite effects on UC vs. CD through nAChRα7. To test this hypothesis, non-specific ligand (nicotine) and several specific ligands (including PNU 282987 (PNU), GTS-21, and MLA, as defined herein below), were used to test their effects on UC-like DSS colitis and CD-like TNBS-colitis in mouse IBD models, the most commonly used animal models in preclinical studies of essentially all therapeutic drugs for IBD.

In some embodiments, the presently disclosed subject matter demonstrates that nAChRα7 agonists PNU 282987 (PNU) (N-[(3R)-1-azabicyclo[2.2.2]oct-3-yl]-4-chlorobenzamide) and 3-(2,4-dimethoxybenzylidene) anabaseine (GTS-21, also referred to herein as DMXB-A) are highly effective therapeutics for UC-like colitis, but worsened CD-like colitis, an observation that is remarkably similar to the smoking effect on human UC vs. CD.

On the other hand, in other embodiments, the presently disclosed subject matter demonstrates that the nAChRα7 antagonist methyllycaconitine (MLS) is a highly effective therapeutic agent for CD-like colitis, but has no effect on UC-like colitis. Again, without wishing to be bound to any one particular theory, it is believed that these agonists and antagonists work by modulating the effects of anti-inflammatory cytokines and pro-inflammatory cytokines. For example, the agonists were capable of stimulating anti-inflammatory cytokines, while suppressing pro-inflammatory cytokines. Further, the presently disclosed subject matter demonstrates that the effect of PNU and MLA is specifically through nAChRα7, since it is not observed in nAChRα7-deficient mice that were induced to have IBD. Therefore, the presently disclosed subject matter not only characterizes the effect of smoking on IBD, but also identifies nAChRα7 as the molecular target for the therapeutic intervention of CD and UC.

More particularly, in some embodiments, the presently disclosed subject matter provides a method for treating an inflammatory bowel disease (IBD) in a subject in need of treatment thereof, the method comprising administering a nicotinic acetylcholine receptor alpha 7 (nAChRα7) antagonist to the subject in an amount effective to modulate an activity of nAChRα7 in at least one cell of the subject, whereby the modulating of the activity of nAChRα7 in the at least one cell treats the IBD in the subject. In some embodiments, the IBD comprises Crohn's disease (CD).

In other embodiments, the presently disclosed subject matter provides a method for treating an inflammatory bowel disease (IBD) in a subject in need of treatment thereof, the method comprising administering an nAChRα7 agonist to the subject in an amount effective to modulate an activity of nAChRα7 in at least one cell of the subject, whereby the modulating of the activity of nAChRα7 in the at least one cell treats the IBD in the subject. In some embodiments, the IBD comprises ulcerative colitis (UC).

The term “effective amount” of an active agent refers to the amount necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, condition, or disorder (e.g., a disease, condition, or disorder related to loss cell function), or one or more symptoms thereof; prevent the advancement of a disease, condition, or disorder; cause the regression of a disease, condition, or disorder; prevent the recurrence, development, onset or progression of a symptom associated with a disease, condition, or disorder; or enhance or improve the prophylactic or therapeutic effect(s) of another therapy. An effective amount of a compound according to the presently disclosed methods can range from, e.g., about 0.001 mg/kg to about 1000 mg/kg, or in certain embodiments, about 0.01 mg/kg to about 100 mg/kg, or in certain embodiments, about 0.1 mg/kg to about 50 mg/kg. Effective doses also will vary, as recognized by those skilled in the art, depending on the disorder treated, route of administration, excipient usage, the age and sex of the subject, and the possibility of co-usage with other therapeutic treatments such as use of other agents. It will be appreciated that an amount of a compound required for achieving the desired biological response may be different from the amount of compound effective for another purpose.

As used herein, the term “agonist” is defined as a substance that stimulates or induces nAChRα7 activity. The term “antagonist” is defined as a substance that blocks or inhibits nAChRα7 activity. In some embodiments, the nAChRα7 receptor agonist or antagonist comprises a small molecule inhibitor including, but not limited to, the exemplary agonists and antagonists provided herein below. nAChRα7 activity, however, can be modulated by any method known to those skilled in the art, including, but not limited to, the use of biologic agents, such as specific antibodies, augmentation of gene expression through gene therapy, or inhibition of gene expression through RNAi.

In particular embodiments, the selective nAChRα7 agonist is selected from the group consisting of: PNU 282987 (N-[(3R)-1-azabicyclo[2.2.2]oct-3-yl]-4-chlorobenzamide), MEM 3454, PH-399733, AR-R1779, SSR180711A (4-bromophenyl 1,4diazabicyclo(3.2.2) nonane-4-carboxylate, monohydrochloride), ABT-418 (3-methyl-5-[(2S)-1-methylpyrrolidin-2-yl]-1,2-oxazole), cocaine methiodide, 3-2,4-dimethoxybenzylidine anabaseine (GTS-21 or DMXB-A), 3-(4-hydroxybenzylidene)anabaseine, 3-(4-methoxybenzylidene)anabaseine, 3-(4-aminobenzylidene)anabaseine, 3-(4-hydroxyl-2-methoxybenzylidene)anabaseine, 3-(4-methoxy-2-hydroxybenzylidene)anabaseine, trans-3-cinnamylidene anabaseine, trans-3-(2-methoxy-cinnamylidene)anabaseine, and trans-3-(4-methoxycinnamylidene)anabaseine.

In other embodiments, the selective nAChRα7 agonist is selected from the group consisting of: N-[(3R)-1-azabicyclo[2.2.2]oct-3-yl]-4-(4-hydroxyphenoxy)benzamide, N-[(3R)-1-azabicyclo[2.2.2]oct-3-yl]-4-(4-acetamidophenoxy)benzamide, N-[(3R)-1-azabicyclo[2.2.2]oct-3-yl]-4-(phenylsulfanylphenoxy)benzamide, N-[(3R)-1-azabicyclo[2.2.2]oct-3-yl]-4-(3-chlorophenylsulphonyl)benzamide, and (1-aza-bicyclo[2.2.2]oct-3-yl) carbamic acid 1-(2-fluorophenyl)-ethyl ester.

Selective nAChRα7 agonists suitable for use with the presently disclosed subject matter are described in the following patent applications: PCT/US2006/022136; PCT/US1998/17850; PCT/EP2007/005724; WO99/62505; WO99/03859; WO97/30998; WO01/36417; WO02/15662; WO02/16355; WO02/16356; WO02/16357; WO02/16358; WO02/17358, U.S. Patent Application Publications Nos. 2006/0166974; 2006/0128676; 2005/0282823; 2005/0165047; 2004/0044026; 2004/0229868, as well as Mazurov A., Hauser T., Miller C. H., “Selective alpha7 nicotinic acetylcholine receptor ligands,” Curr Med Chem 13:1567-1584 (2006), each of which is incorporated herein by reference in its entirety.

In certain embodiments, the nAChRα7 agonist is selected from the group consisting of an nAChRα7-specific antibody, an nAChRα7-specific inhibitory RNA, or combinations thereof.

In some embodiments, the selective nAChRα7 antagonists include α-bunngarotoxin and methyllycaconitine (MLA). Further examples of selective nAChRα7 antagonists are described in Mazurov A., Hauser T., Miller C. H., “Selective alpha7 nicotinic acetylcholine receptor ligands,” Curr Med Chem 13:1567-1584 (2006), which is incorporated herein by reference in its entirety. In certain embodiments, the nAChRα7 antagonist is selected from the group consisting of an nAChRα7-specific antibody, an nAChRα7-specific inhibitory RNA, or a combination thereof.

In other embodiments, the nAChRα7 receptor ligands include, but are not limited to, diazabicycloalkane derivatives, for example, as described in WO2005/028477; spirocyclic quinuclidinic ether derivatives, for example, as described in WO2005/066168; fused bicycloheterocycle substituted quinuclidine derivatives, for example, as described in U.S. Patent Application Publication Nos. US2005/0137184; US2005/0137204; and US2005/0245531; 3-quinuclidinyl amino-substituted biaryl derivatives, for example, as described in WO2005/066166; 3-quinuclidinyl heteroatom-bridged biaryl derivatives, for example, as described in WO2005/066167; and amino-substituted tricyclic derivatives, for example, as described in WO2005/077899, each of which is incorporated by reference in its entirety.

Throughout the specification and claims, a given chemical formula or name shall encompass all tautomers, congeners, and optical- and stereoisomers, as well as racemic mixtures where such isomers and mixtures exist.

In some embodiments, the treating comprises preventing the development of the IBD in the subject or preventing the progression of the IBD in the subject. The terms “treating” or “treatment” and grammatical derivations thereof, are intended to encompass the therapy, management, prophylaxis, and cure of a disease state or condition.

The term “subject” refers to an organism, tissue, or cell. A subject can include a human subject for medical purposes, such as diagnosis and/or treatment of an existing condition or disease or the prophylactic treatment for preventing the onset of a condition or disease, or an animal subject for medical, veterinary purposes, or developmental purposes. A subject also can include sample material from tissue culture, cell culture, organ replication, stem cell production and the like. Suitable animal subjects include mammals and avians. The term “mammal” as used herein includes, but is not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. The term “avian” as used herein includes, but is not limited to, chickens, ducks, geese, quail, turkeys, and pheasants. Preferably, the subject is a mammal or a mammalian cell. More preferably, the subject is a human or a human cell. Human subjects include, but are not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. A subject also can refer to cells or collections of cells in laboratory or bioprocessing culture in tests for viability, differentiation, marker production, expression, and the like.

In some embodiments, the subject is a rodent, e.g., a mouse, which has been chemically induced to be models of IBD. In particular embodiments, the mouse model is one or more of the following: (a) a mouse that has been exposed to dextran sulfate sodium (DSS) to induce an ulcerative colitis-like condition; (b) a mouse that has been exposed to trinitrobenzene sulfonic acid (TNBS) to induce a Crohn's disease-like condition; (c) a mouse that is nAChRα7-deficient that has been induced to have colitis; or (d) a mouse that is deficient in two glutathione peroxidases (GPX), Gpx1 and Gpx2, [Gpx1/2-double knockout (DKO) mice], and subsequently develop ileocolitis.

In other embodiments, the presently disclosed methods are directed to eukaryotic cells, preferably mammalian cells, and more preferably human cells. The cells, or a cell line, can be obtained commercially or can be isolated from mammalian tissue. Cells suitable for use with the presently disclosed methods also can be present as part of tissues or organ preparations. Cells, without limitation, can be obtained from rat, cat, horse, mouse, hamster, chicken, sheep, goat, pig, cow, rabbit, non-human primates, and humans.

In other embodiments, the presently disclosed subject matter provides a method for modulating an activity of nAChRα7 in at least one immune cell type, the method comprising contacting the at least one immune cell type with an nAChRα7 antagonist or a nAChRα7 agonist in an amount effective to modulate the activity of nAChRα7 in the at least one immune cell type.

As used herein, the phrase “immune cells” is meant to indicate those cells that are functional in the mammalian immune response, including the cell-mediated immune response and the humeral immune response. The phrase includes, but is not limited to, lymphocytes, antigen-specific cytotoxic T-lymphocytes, neutrophils, peripheral blood mononuclear cells (PBMC), macrophages, and natural killer cells.

In yet other embodiments, the presently disclosed subject matter provides a method for reducing the risk of developing colorectal cancer in a subject having a chronic gastrointestinal tract inflammation, the method comprising administering an nAChRα7 antagonist or an nAChRα7 agonist to the subject in an amount effective to modulate an activity of nAChRα7 in one or more immune cells of the gastrointestinal tract, whereby modulating the activity of nAChRα7 alters an inflammatory response in the one or more immune cells of the gastrointestinal tract, thereby reducing the risk of developing colorectal cancer.

In further embodiments, the presently disclosed subject matter provides a method for identifying a compound or agent that modulates an activity of nAChRα7 in at least one cell expressing nAChRα7, the method comprising: (i) contacting the at least one cell expressing nAChRα7 with a candidate compound or agent; (ii) determining the activity of nAChRα7 in the at least one cell expressing nAChRα7 that has been contacted with the candidate compound or agent; (iii) determining the activity of nAChRα7 in at least one control cell expressing nAChRα7 that has not been contacted with the candidate compound or agent; and (iv) comparing the activity of nAChRα7 in the at least one cell that has been contacted with the candidate compound or agent to the activity of nAChRα7 in the at least one control cell; wherein a difference in the activity of nAChRα7 in the at least one cell that has been contacted with the candidate compound or agent and the at least one control cell identifies a candidate compound or agent that modulates the activity of nAChRα7 in at least one cell.

In yet further embodiments, the presently disclosed subject matter provides a method for predicting a therapeutic effect of administering a modulator of nAChRα7 expression to a subject afflicted with IBD resulting from an abnormal level of gene or protein expression of nAChRα7, wherein the modulator of nAChRα7 expression is an nAChRα7 antagonist or an nAChRα7 agonist, the method comprising: (a) measuring a level of gene or protein expression of nAChRα7 in a tissue or cell of the subject before administering the nAChRα7 modulator; (b) administering an nAChRα7 modulator to the subject in an amount effective to alter an nAChRα7 gene or protein expression level in a tissue or cell of the subject; (c) measuring a level of gene or protein expression of nAChRα7 in a tissue or cell from the subject after administering the nAChRα7 modulator; and (d) determining an alteration in the level of gene or protein expression of nAChRα7 in the tissue or cell after administering the nAChRα7 modulator from the level of gene or protein expression in the tissue or cell before administering the nAChRα7 modulator; wherein an alteration in the level of nAChRα7 gene or protein expression in the tissue or cell predicts a therapeutic effect of the modulator of nAChRα7 expression to a subject afflicted with IBD.

In some embodiments, nAChRα7 is overexpressed to abnormal levels in the subject afflicted with IBD, and a decrease in gene or protein expression level predicts a positive response to administering the nAChRα7 modulator. In other embodiments, nAChRα7 is downregulated to abnormal expression levels in the subject afflicted with IBD, and an increase in gene or protein expression level predicts a positive response to administering the nAChRα7 modulator.

In some embodiments, the alteration in gene expression level is determined using one or more methods selected from the group consisting of Northern blotting, RT-PCR, real-time RT-PCR, in-situ hybridization, and microarrays. In other embodiments, the alteration in protein expression level is determined using one or more methods selected from the group consisting of Western Blotting, ELISA, mass spectrometry, immunohistochemistry, and protein arrays.

Accordingly, in some embodiments, the presently disclosed methods can be used to diagnose, for the prognosis, or the monitoring of a disease state or condition. As used herein, the term “diagnosis” refers to a predictive process in which the presence, absence, severity or course of treatment of a disease, disorder or other medical condition is assessed. For purposes herein, diagnosis also includes predictive processes for determining the outcome resulting from a treatment. Likewise, the term “diagnosing,” refers to the determination of whether a subject exhibits one or more characteristics of a condition or disease. The term “diagnosing” includes establishing the presence or absence of, for example, a target antigen or reagent bound targets, or establishing, or otherwise determining one or more characteristics of a condition or disease, including type, grade, stage, or similar conditions. As used herein, the term “diagnosing” can include distinguishing one form of a disease from another. The term “diagnosing” encompasses the initial diagnosis or detection, prognosis, and monitoring of a condition or disease. Further, the term “monitoring,” such as in “monitoring the course of a disease or condition,” refers to the ongoing diagnosis of samples obtained from a subject having or suspected of having a disease or condition. The term “prognosis,” and derivations thereof, refers to the determination or prediction of the course of a disease or condition. The course of a disease or condition can be determined, for example, based on life expectancy or quality of life. “Prognosis” includes the determination of the time course of a disease or condition, with or without a treatment or treatments. In the instance where treatment(s) are contemplated, the prognosis includes determining the efficacy of a treatment for a disease or condition. As used herein, the term “risk” refers to a predictive process in which the probability of a particular outcome is assessed.

Accordingly, the presently disclosed subject matter includes monitoring nAChRα7 gene expression and/or protein levels as a prognostic measurement of therapeutic response in patients with IBD. The effect on nAChRα7 gene expression and/or protein levels in response to therapeutic can provide a means for determining whether the patient is responding to the treatment. In cases where nAChRα7 is over-expressed, such as in patients with Crohn's disease (CD), a decrease in gene/protein expression levels should indicate a positive response to treatment. In cases where nAChRα7 is down-regulated, such as in patients with ulcerative colitis, an increase in gene/protein expression levels should indicate a positive response to treatment. Methods of detecting gene expression levels can be done by any method known to those skilled in the art, including, but not limited to, those disclosed immediately hereinabove. Such methods can use RNA or protein from any tissue sample of interest, such as IBD tissue biopsies, fresh or archival surgical specimens, circulating cells present in the blood, serum, urine, and cell lines.

### II. Pharmaceutical Compositions, Kits, and Administration

In another aspect, the present disclosure provides a pharmaceutical composition including one or more nAChRα7 antagonists and/or nAChRα7 agonists alone or in combination with one or more additional therapeutic agents in admixture with a pharmaceutically acceptable excipient. One of skill in the art will recognize that the pharmaceutical compositions include the pharmaceutically acceptable salts of the compounds described above.

More particularly, in some embodiments, the presently disclosed subject matter provides a pharmaceutical composition comprising a specific agonist or a specific antagonist of a nicotinic acetylcholine receptor alpha 7 (nAChRα7) in an amount effective to modulate the function of nAChRα7 to treat or prevent an inflammatory bowel disease (IBD) in a subject in need of treatment thereof. In some embodiments, the pharmaceutical composition comprises a therapeutically effective amount of an nAChRα7 agonist/antagonist formulated together with one or more non-toxic pharmaceutically acceptable carriers, adjuvants, or excipients. The compositions described therein, or pharmaceutically acceptable addition salts or hydrates thereof, can be delivered to a subject so as to avoid or reduce undesirable side effects according to the presently disclosed methods using a wide variety of routes or modes of administration. The pharmaceutical compositions can be formulated for oral administration in solid or liquid form.

A variety of ingredients known by those of skill in the art that do not interfere with the function of the pharmaceutically acceptable carrier may optionally be included in the pharmaceutical composition in effective amounts. Generally, lubricants, binders, gelatin, and/or disintegrants are suitable. Other optional ingredients include buffers, preservatives, tonicity adjusting agents, antioxidants, polymers for adjusting viscosity, or for use as extenders, and excipients, and the like. Other conventional additives known in those having ordinary skill in the pharmaceutical arts include, but are not limited to, humectants, emollients, stabilizers, and dyes, and may be used providing the additives do not interfere with the therapeutic properties of the pharmaceutical composition. The pharmaceutical compositions are readily prepared using methods generally known in the pharmaceutical arts. The compounds described or pharmaceutically acceptable salts and/or hydrates thereof can be administered singly, in combination with other presently disclosed compounds, and/or in combination with other therapeutic agents for treatment or prophylaxis of IBD.

In other embodiments, the presently disclosed subject matter provides a kit comprising the presently disclosed pharmaceutical compositions. The kit includes at least one pharmaceutical composition comprising the agonist or antagonist, or a salt thereof. The kit also includes a container for containing the compositions. In some embodiments, the kit further comprises directions for administering to the subject the at least one pharmaceutical composition comprising the agonist or antagonist, or a salt thereof.

Suitable routes of administration include, but are not limited to, inhalation, transdermal, oral, rectal, transmucosal, intestinal, and parenteral administration, including intramuscular, subcutaneous, and intravenous injections. In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

The compounds according to the disclosure are effective over a wide dosage range. For example, in the treatment of adult humans, dosages from 0.01 to 1000 mg, from 0.5 to 100 mg, and from 1 to 50 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Depending upon the particular condition, or disease state, to be treated or prevented, additional therapeutic agents, which are normally administered to treat or prevent that condition, may be administered together with the agonists and/or antagonists of this disclosure. Accordingly, additional agents may be combined with the presently disclosed nAChRα7 antagonists and/or nAChRα7 agonists in a pharmaceutical composition. These additional agents may be administered separately, as part of a multiple dosage regimen, from the inhibitor-containing composition. Alternatively, these agents may be part of a single dosage form, mixed together with the inhibitor in a single composition.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Gene Expression Profiles Show Distinct Expression Patterns in IBD

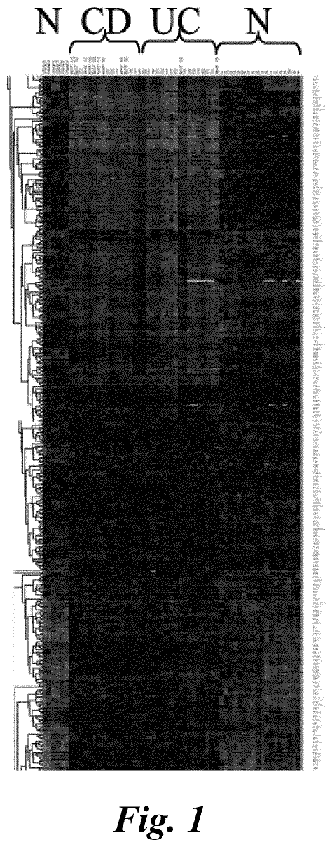

Peripheral blood and colonic biopsies were obtained from 17 patients, mean age 46.53 (95% Cl, 39.65-53.41) with documented IBD (9 CD and 8 UC). The cohort also included 17 age- and sex-matched unaffected healthy controls. Neutrophils and peripheral blood mononuclear cells (PBMCs) were isolated, and total RNA was extracted. Cy-labeled cDNA was hybridized to genome-scale microarrays containing probes for 21,329 genes. After data normalization, prior to associative analysis, approximately 9,500 genes for each sample were expressed three (3) standard-deviations above the background. Hierarchical clustering demonstrated the robustness of the analysis method for sample class categorization, suggesting that distinct clusters of gene-expression profiles exist within each subgroup (see FIG. 1). Discriminant functional analysis (DFA) was used to identify genes whose expression in immune cells maximally distinguished the cohort. Alex P., Szodoray P., Knowlton N., Dozmorov I. M., Turner M., Frank M. B., Arthur R. E., Willis L., Flinn D., Hynd R. F., Carson C., Kumar A., El-Gabalawy H. S., Centola M., “Multiplex serum cytokine monitoring as a prognostic tool in rheumatoid arthritis,” Clin Exp Rheumatol 25:584-592 (2007).

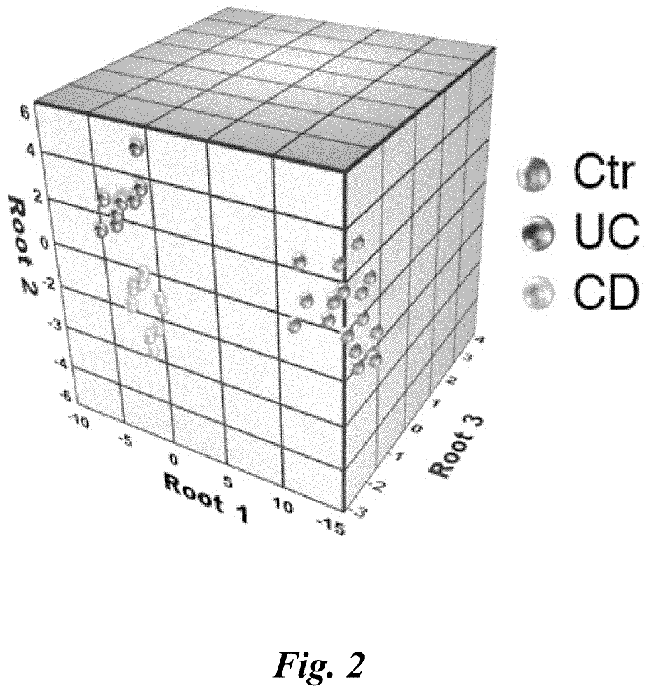

Of the 96 differentially expressed genes, 10 were selected by a stringent DFA as having the highest power for class discrimination between patients and controls. Intriguingly, on a 3D plot of DFA root values, UC and CD patients and controls were grouped into three distinct positions, suggesting that this is directly proportional to intergroup gene expression variation and intragroup heterogeneity in overall gene expression and disease pathology (FIG. 2). nAChR α7 is one of the 10 differentially expressed genes.

### Example 2

### Differential nAChRα7 Expression Patterns Identified in CD Vs. UC

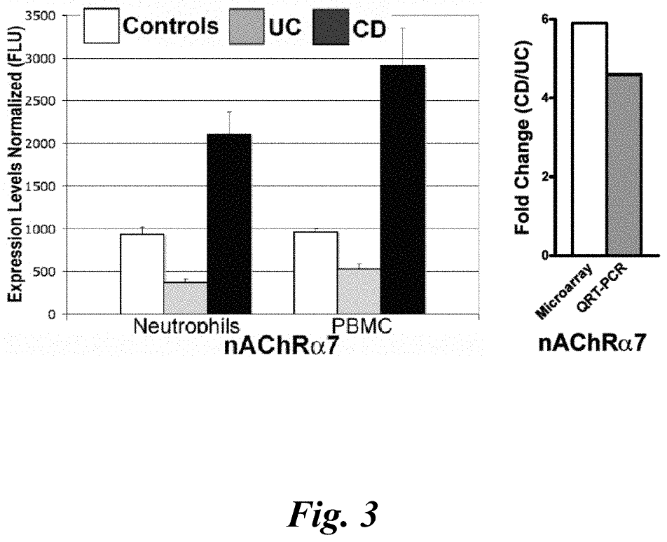

Compared to controls, nAChRα7 was significantly upregulated in neutrophils and PBMCs from patients with CD, while downregulated in both neutrophils and PBMCs from patients with UC, as revealed by gene microarray analysis (FIG. 3, left panel). The expression level of nAChRα7 in CD relative to UC was validated by QRT-PCT (FIG. 3, right panel: the fold change of CD vs. UC is statistically the same. Since nicotine is the major active component in smoking and nAChRα7 is the only nAChR that is expressed differentially in UC vs. CD, these data suggested that nAChRα7 might be a key player in modulating the distinctive pro-inflammatory effect on CD and anti-inflammatory effect on UC in smokers vs. non-smokers.

### Example 3

### Distinct Immunomodulatory Profiles of Nicotine Mediated Through nAchRα7 in CD Vs. UC

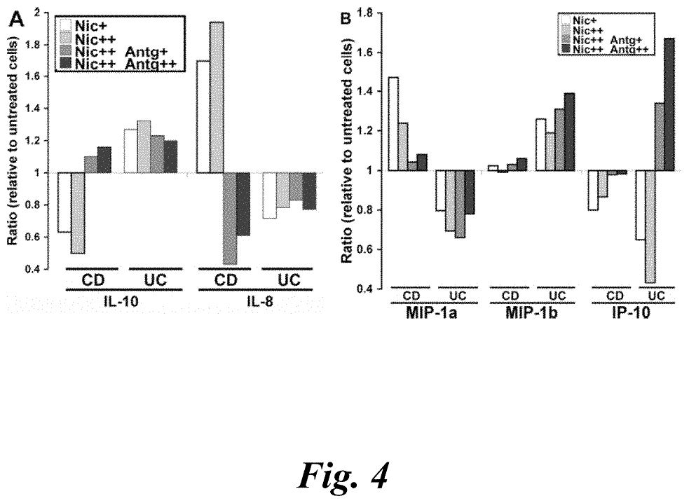

Given the unique differential expression of nAchRα7 in CD vs. UC and healthy (FIG. 3), preliminary studies were performed to investigate the functional contribution of nAchRα7 and its immunomodulatory profiles in CD vs. UC. Although IBD has been largely investigated as a T-cell mediated disease, cognate gut microbe/B/T cell interactions are important in the pathogenesis of the disease. Immortalized (EBV-transformed) B lymphocytes of patients with UC, CD, and unaffected controls were investigated to evaluate the immunmodulatory profiles of nAchRα7. Stimulation and inhibition studies with nicotine (2 μM and 20 μM) and nAchRα7 selective antagonists α-bungarotoxin (Sigma, St. Louis) (2 nM and 20 nM) were performed on cells cultured and maintained at a final concentration of 1×10⁶ cells. A sandwich immunoassay-based protein array system (a biometric sandwich ELISA that contains dyed microspheres conjugated with a monoclonal antibody specific for a target protein), as previously described, Alex P., Szodoray P., Knowlton N., Dozmorov I. M., Turner M., Frank M. B., Arthur R. E., Willis L., Flinn D., Hynd R. F., Carson C., Kumar A., El-Gabalawy H. S., Centola M., “Multiplex serum cytokine monitoring as a prognostic tool in rheumatoid arthritis,” Clin Exp Rheumatol 25:584-592 (2007); Nakayama T., Hieshima K., Nagakubo D., Sato E., Nakayama M., Kawa K., Yoshie O., “Selective induction of Th2-attracting chemokines CCL17 and CCL22 in human B cells by latent membrane protein 1 of Epstein-Barr virus,” J Virol 78:1665-1674 (2004), was used to measure the levels of 22 cellular, cytotoxic, humoral cytokines and chemokines released into the media following stimulation at various time points (24, 48, 72, 96, and 120 hours) (FIG. 4). Validation of the multiplex assays was performed using single protein ELISAs. Of these 22 variables, significant changes in the secretion of immune mediators from EBV transformed lymphocytes between unaffected controls and IBD were only evident in five cytokines/chemokines (FIG. 4) (reproduced in two separate experiments). This profile is typicl of the reported “low Th1, high Th2 chemotactic” immune production of B lymphoblastic cells. Nakayama T., Hieshima K., Nagakubo D., Sato E., Nakayama M., Kawa K., Yoshie O., “Selective induction of Th2-attracting chemokines CCL17 and CCL22 in human B cells by latent membrane protein 1 of Epstein-Barr virus,” J Virol 78:1665-1674 (2004); Burdin N., Peronne C., Banchereau J., Rousset F., “Epstein-Barr virus transformation induces B lymphocytes to produce human interleukin 10,” J Exp Med 177:295-304 (1993); Vockerodt M., Pinkert D., Smola-Hess S., Michels A., Ransohoff R. M., Tesch H., Kube D., “The Epstein-Barr virus oncoprotein latent membrane protein 1 induces expression of the chemokine IP-10: importance of mRNA half-life regulation,” Int J Cancer 114:598-605 (2005).

Pretreatment with varying doses of nicotine for 120 hours demonstrated significant production of proinflammatory cytokine/chemokine IL-8 and MIP-1 in CD relative to UC, and a signficant inhibition of anti-inflammatory cytokine IL-10 production in CD relative to CD (FIG. 4A), suggesting that nicotine has distinct pro-inflammatory and chemotactic effects in CD. The effects of nicotine in the suppression of IL-10 in CD (FIG. 4A) also suggests a potential mechanism of which also is agreement with the well-documented chronic CD-like ileocolitis that develops in gene targeted IL-10 knockout mice, and by the reported therapeutic efficacy of IL-10 in several anmial models of colitis. Kuhn R., Lohler J., Rennick D., Rajewsky K., Muller W., “Interleukin-10-deficient mice develop chronic enterocolitis 2,” Cell 75:263-274 (1993); Ribbons K. A., Thompson J. H., Liu X., Pennline K., Clark D. A., Miller M. J., “Anti-inflammatory properties of interleukin-10 administration in hapten-induced colitis,” Eur J Pharmacol 323:245-254 (1997). Since α-bungartoxin significantly reversed the effects of nicotine at 100% in CD, these data also explicates the pro-inflammatory effects in CD through a nAChRα7-mediated mechanism. Similar studies to investigate the nAChRα7-mediated cytokine profiles in freshly isolated lymphocytes and neutrophils are ongoing. The above findings strongly indicate a critical role of nAChRα7 in modulating nicotine effect and the significant potential of nAChRα7 as a therapeutic target for CD therapy.

Pre-treatment with nicotine or nAChRα7-specific agonists prevents the development of DSS-induced “UC-like” mouse colitis (disease phenotype resembles human UC), while significantly worsens the “CD-like” TNBS-induced colitis (disease phenotype resembles human CD).

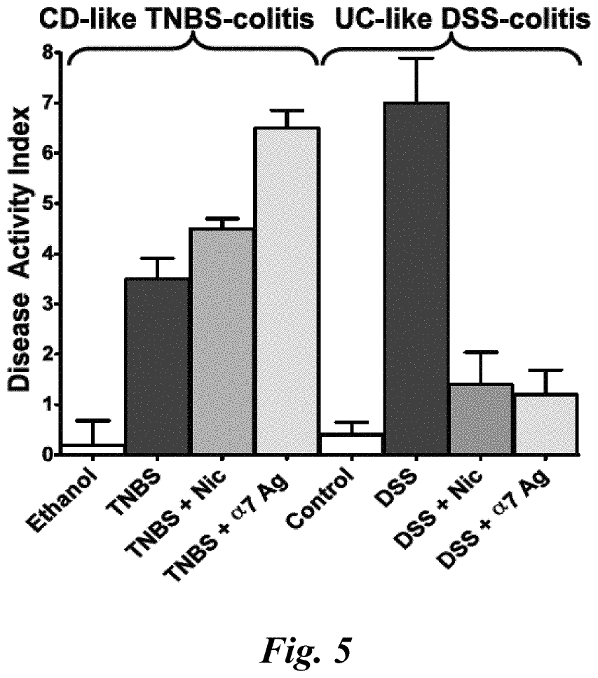

To obtain direct evidence that nAChRα7 is the master modulator to orchestrate the opposite effects of nicotine on the courses of CD and UC; a series of experiments were conducted using well-established chemically-induced mouse IBD models (DSS- and TNBS-colitis) (FIGS. 5-13), which have been extensively used in preclinical studies of therapeutic drugs for IBD. For CD-like mouse model, BALB/c mice were used for the induction of DSS colitis. For CD-like mouse model, BALB/c mice were used for the induction of TNBS-colitis. C57/B6 or BALB/c mice, 6-8 weeks old, were pretreated (daily peritoneal injection) for one week with either nicotine (a non-specific ligand of nAChRα7; 6 mg/kg) or a nAChRα7-specific agonist (PNU; 8.3 mg/kg). Controls were mice without the pre-treatments (a total of >10 mice in each treatment group). The mice were then induced to develop either CD-like colitis with TNBS (600 μg) or UC-like colitis with DSS (2.5%). As shown in FIG. 5 for the disease activity index (DAI) in the acute colitis models, pretreatment with nicotine or PNU for five days greatly worsens the CD-like TNBS-colitis. In sharp contrast, however, nicotine and PNU prevents DSS-induced UC-like colitis, with the PNU being slightly more potent. Examination of colon morphology and histology further confirmed the changes in DAI (data not shown). The results of these preclinical studies in mouse model accurately mimic the clinically well-characterized opposite effects of smoking on human IBD in that smoking is detrimental to CD while beneficial in UC. These data established: (1) the role of nicotine in the pathogenesis of CD and therapeutic benefit of UC; and (2) nAChRα7 as master switch that controls the distinctive responses of nicotine (smoking) to be either pro-inflammatory (as in CD) or anti-inflammatory (as in UC). Agonist GTS-21 has a similarly effect of preventing development of DSS-induced CD-like colitis.

### Example 4

### Agonists of nAChRα7 are Highly Effective in the Therapy of Chronic “UC-Like” DSS Colitis

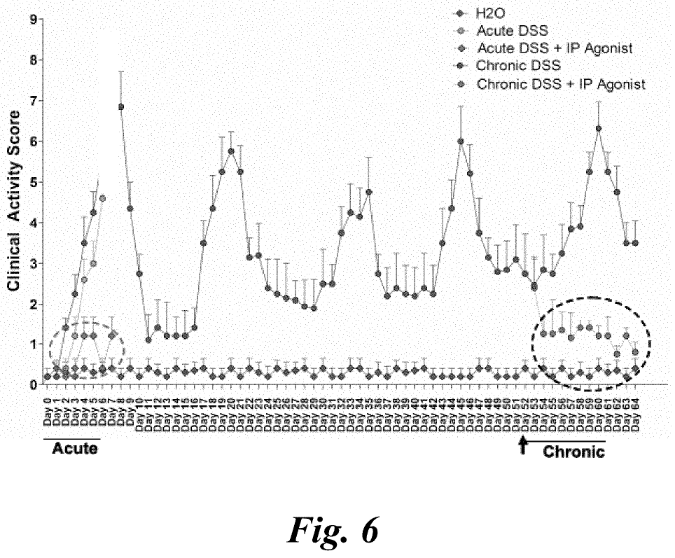

nAChRα7 agonists (PNU and GTS-21) are capable of not only preventing DSS-induced colitis in the acute colitis model (FIG. 5), they also can effectively reverse the disease course in chronic colitis model (FIG. 6), which is considered to be much more resembling human UC. Induction of chronic UC-like colitis was described in the legends of FIG. 6. As shown in FIG. 6, in which effect of PNU on DAI of both acute and chronic colitis models were shown. PNU treatments essentially eliminated the inflammation in both models.

### Example 5

### Antagonist of nAChRα7, MLA, is Highly Effective in Preventing CD-Like TNBS Colitis

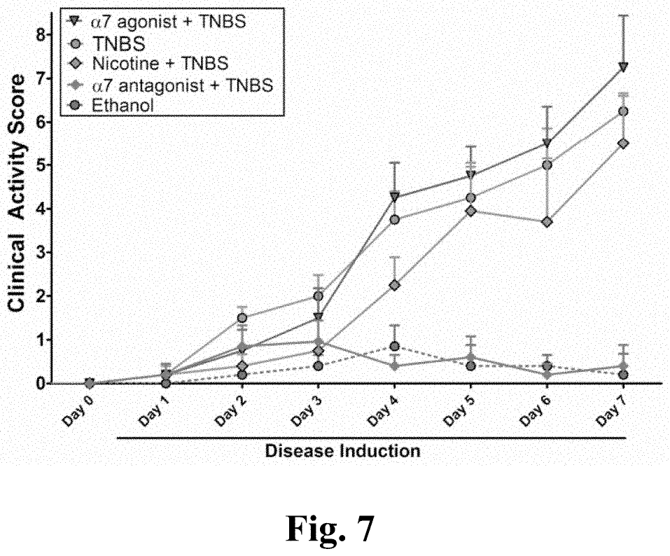

Based on the observation that nAChRα7 is down-regulated in UC and upregulated in CD, without wishing to be bound to any one particular theory, it is hypothesized that nAChRα7-specific antagonists will inactivate the function of nAChRα7, and thereby have therapeutic effects on CD. Therefore, the effect of nAChRα7-specific antagonist MLA on the CD-like TNBS-colitis was tested using BALB/c mice. Indeed, as shown in FIG. 7, it was found that the 5 days pretreatment of mice with nAChRα7-specific antagonist MLA could efficiently prevent the mice from developing TNBS-induced colitis (FIG. 7). Importantly, nAChRα7 agonist PNU treatment not only has no effect in the colitis development, but also worsens the disease. Nicotine, an agonist that is less specific to nAChRα7, but effective in preventing UC-like DSS-colitis, has no effect on the disease development either (FIG. 5). These results mimic the smoking effects in UC and CD. Taken together with the data shown in FIGS. 5 and 6, these results suggest that nAChRα7-specific antagonist (MLA) has effective therapeutic benefit in CD, while nAChRα7-specific agonists (PNU and GTS) have effective therapeutic effects in UC. The highly therapeutic effect of MLA on the CD-like chronic TNBS-colitis (using BALB/c mice) also has been demonstrated (data not shown), with efficacy similar to that of PNU on the UC-like DSS-colitis (FIG. 6).

### Example 6

### Antagonists of nAChRα7 are Highly Effective in the Therapy of Chronic “CD-Like” TNBS Colitis

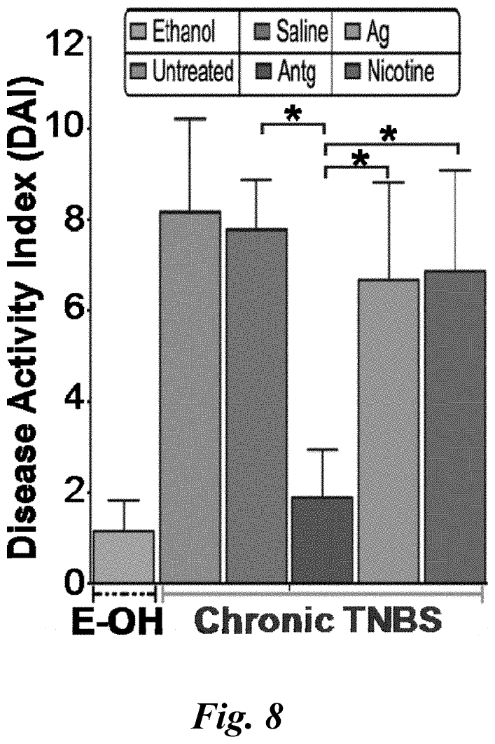

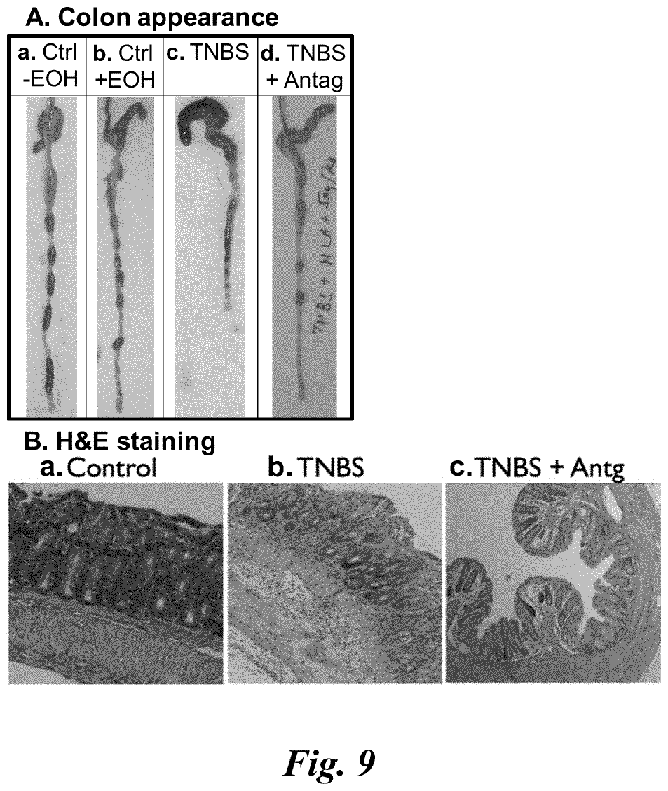

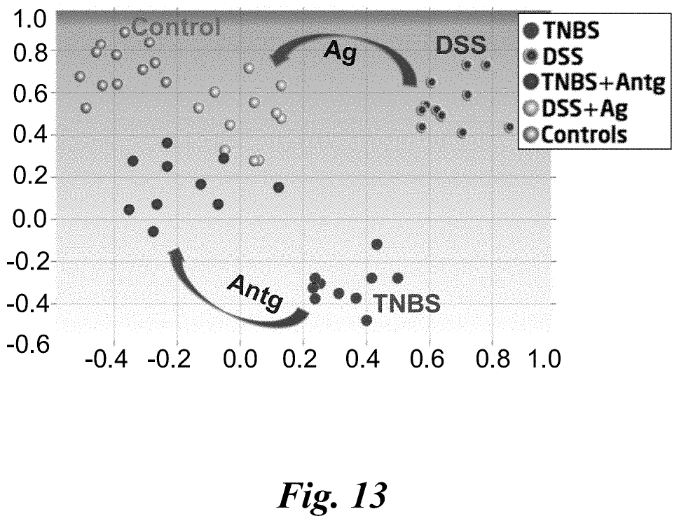

nAChRα7 antagonists (MLA) are capable of not only preventing acute TNBS-colitis, FIG. 7), they also can effectively reverse the disease course in chronic TNBS colitis model (FIG. 8), which is considered to be much more resembling human CD. Induction of chronic CD-like colitis was described previously (Alex et al, 2009 IBD, 15(3), 341-352). While agonist treatment had no effect on the disease activity, antagonist treatments essentially eliminated the inflammation in both models. The mice appeared normal and indistinguishable from the ethanol treated control mice. Histological analysis by H&E staining (FIG. 9) further demonstrated the highly effective therapeutic effect of the nAChRα7-specific antagonist on CD-like colitis. The therapeutic effects also can be seen biochemically, as treatments, either by agonist for DSS-colitis or by antagonist for TNBS-colitis, dramatically altered the cytokine profiles of from disease states toward normal healthy state (FIG. 13).

### Example 7

### Agonists PNU and Antagonist MLA have No Therapeutic Effect in nAChRα7-Knockout Mice that were Induced to have Colitis, Further Demonstrating that the Drugs are Specific to nAChRα7 Receptor

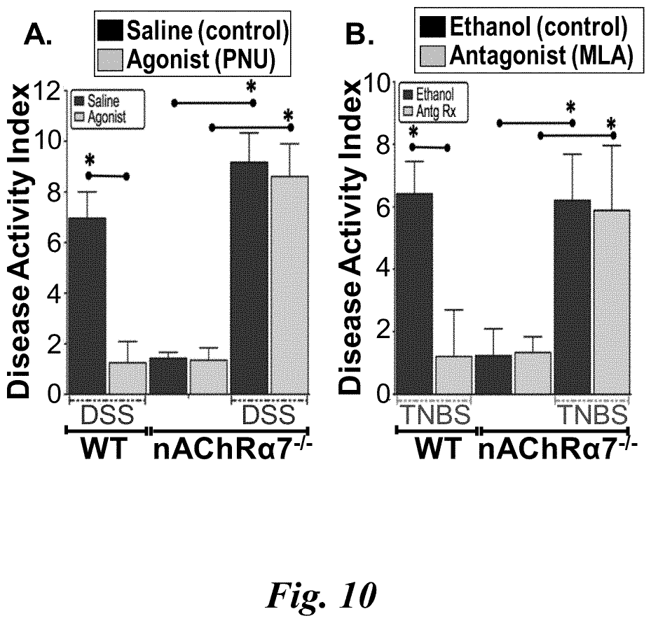

Although the presently disclosed data from both cell models and mouse colitis models clearly identified nAChRα7 as a key to the pathogenesis of CD in response to nicotine, the possibility that other nAChRs are not involved cannot be completely excluded since the specificity of the nAChRα7 agonist/antagonist used may not be limited to only nAChRα7. To exclude this possibility, nAChRα7 agonist and antagonist were tested on nAChRα7-knockout mice (nAChRα7-KO), in which development of chemically-induced colitis will not be affected by the agents if they are only specific to nAChRα7. It was found that nAChRα7-KO mice are more sensitive to DSS- or TNBS-induced colitis (FIG. 10). More importantly, neither agonist (PNU) nor antagonist (MLA) was efficacious as a therapeutic agent in DSS- or TNBS-induced colitis in nAChRα7-KO mice (FIG. 10). These data demonstrate that the target of these drugs is specific to nAChRα7.

### Example 8

### The Anti-Inflammatory Effect of nAChRα7 Agonist on DSS-Colitis is Completely Abolished by Vagotomy, Providing the First Definitive In Vivo Evidence that Vagus Nerve Plays a Critical Role in nAChRα7-Mediated Anti-Inflammatory Pathway in IBD

Efferent signals from vagus nerve, which can be controlled by brain networks, inhibit cytokine production via the cholinergic anti-inflammatory pathway that depends on the nAChRα7 on non-neuronal cells, particularly macrophages. Through this pathway, stimulation of vagus nerve prevents damaging effects of cytokine release in various inflammatory diseases, including sepsis, endotoxemia, ischemia, hemorrhagic shock, arthritis, and postoperative ileus. Although it has been shown that vagotomized mice are more sensitive to DSS- and TNBS-induced colitis, there is no direct experimental evidence specifically linking the vagus nerve and nAChRα7 in modulating IBD. Since data presented earlier clearly demonstrated the specific involvement of nAChRα7 in the anti-inflammatory effects on UC and the pathogenesis of CD, it was further determined if this nAChRα7-specific effect is dependent on the vagus nerve.

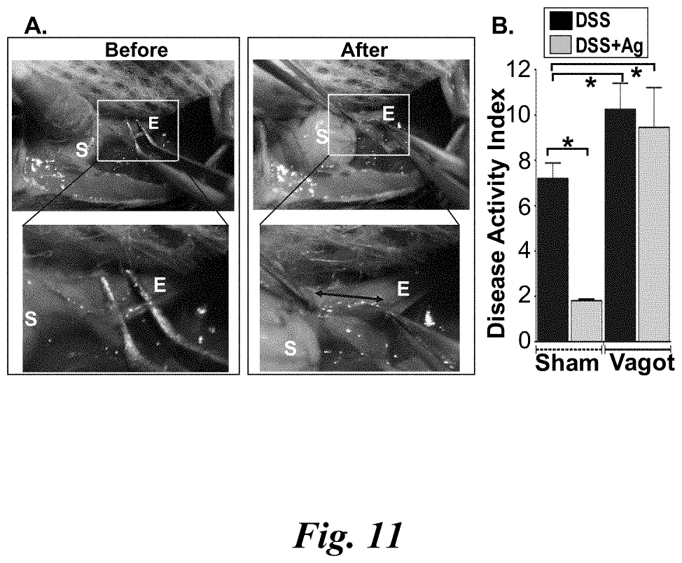

Vagotomy was performed at the left vagal branch (approximately 5-10 mm) at the gastroesophageal junction of the subdiaphragmatic vagus of anesthetized C57B/6 mice (7 weeks old) FIG. 11A). Vagotomized mice were given 7 days to recover, and then administered nAChRα7 agonist by IP for 5 days before DSS treatment (7 days) to induce colitis. As shown in FIG. 11B, vagotomized mice completely lost the protective effect of nAChRα7 agonist on colitis. These data suggest that the nAChRα7-mediated anti-inflammatory effects on IBD are entirely dependent on the functional vagus nerve.

### Example 9

### Agonists PNU is Highly Effective in Treating Colitis Mediated by GPX 1/2 DKO

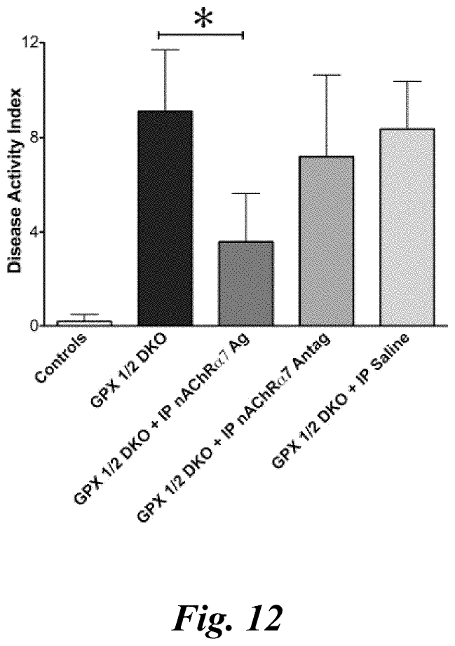

Since most of the prior in-vivo studies were performed in experimental colitis models, the hypothesis that nAChRα7 agonist is effective in treating colitis mediated by a genetic defect was tested. Mice deficient in two glutathione peroxidases (GPX), Gpx1 and Gpx2, [Gpx1/2-double knockout (DKO) mice] spontaneously develop ileocolitis on a mixed C57BL/6 and 129S1/SvJ (B6.129) genetic background. Lee D. H., Esworthy R. S., Chu C., Pfeifer G. P., Chu F. F., “Mutation accumulation in the intestine and colon of mice deficient in two intracellular glutathione peroxidases,” Cancer Res. 66(20):9845-51 (Oct. 15, 2006). A subgroup of B6.129 Gpx1/2-DKO mice develop ileocolonic inflammation by 6 to 8 weeks, and ileocolonic tumors by 6 to 9 months. While controls (from mixed C57BL/6 and 129S1/SvJ (B6.129) genetic background) do not develop signs of colitis, GPX 1/2 DKO mice were found to develop severe colitis with a clinical activity score of approximately 9 (FIG. 12). Once signs of colitis were immediately evident, both nAChRα7 agonists and nAChRα7 antagonists were administered to Gpx1/2-DKO mice. In GPX 1/2 DKO mice that were treated with nAChRα7 agonist, the development of disease (colitis) was significantly decreased, suggesting that nAChRα7 agonist PNU is highly effective in treating colitis mediated by GPX 1/2 DKO (FIG. 12). GPX 1/2 DKO mice treated with nAChRα7 antagonist or saline, however, did not demonstrate significant changes in colitis scores. These data demonstrate that nAChRα7 agonists are highly effective in treating both chemical and genetic models of colitis.

### Example 10

### PNU has No Observable Adverse Effects on Major Organs and Behavior of Mice

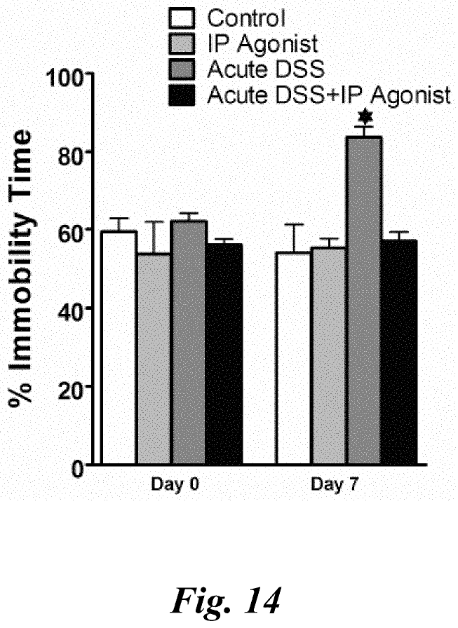

At the dose used in the preclinical studies, the PNU drug exhibited no adverse effect on mice. All major organs, including heart, kidney, liver, small and large intestines, spleen, look normal by appearance. Forced swimming test: Mice were forced to swim in a cylindrical container for 6 minutes as described by Porsolt R. D., Bertin A., Jalfre M., “Behavioral despair in mice: a primary screening test for antidepressants,” Arch Int Pharmacodyn Ther 229:327-336 (1977), to assess if the drug alters behavior of mice (mobility as a readout). Zomkowski A. D., Santos A. R., Rodrigues A. L., “Evidence for the involvement of the opioid system in the agmatine antidepressant-like effect in the forced swimming test,” Neurosci Lett 381:279-283 (2005). As shown in FIG. 14, no significant behavior change of mice was observed with or without the drug treatment. Only mice with DSS-induced colitis displayed significant immobility. Treatment with agonist PNU prevented colitis and thereby completely reversed the immobility of DSS-mice. This result suggests that nAChRα7 agonist does not have adverse effect on the behavior of the mice studied. Other studies of these nAChRα7-specific drugs, including toxicity and pharmacokinetics, are being investigated.

In summary, the presently disclosed compounds provide a single therapeutic target, nAChRα7, for both CD and UC: While agonists of nAChRα7 (such as PNU and GTS-21) are highly effective therapeutic drugs for UC, antagonists of nAChRα7 (such as MLA) have significant therapeutic potential for CD.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Xavier R J, Podolsky D K. Unravelling the pathogenesis of inflammatory bowel disease. Nature 2007; 448:427-434.
Loftus E V, Jr. Clinical epidemiology of inflammatory bowel disease: Incidence, prevalence, and environmental influences. Gastroenterology 2004; 126:1504-1517.
Shanahan F. Review article: colitis-associated cancer—time for new strategies 1. Aliment Pharmacol Ther 2003; 18 Suppl 2:6-9.
Campbell B J, Yu L G, Rhodes J M. Altered glycosylation in inflammatory bowel disease: a possible role in cancer development. Glycoconj J 2001; 18:851-858.
Birrenbach T, Bocker U. Inflammatory bowel disease and smoking: a review of epidemiology, pathophysiology, and therapeutic implications. Inflamm Bowel Dis 2004; 10:848-859.
Rubin D T, Hanauer S B. Smoking and inflammatory bowel disease. Eur J Gastroenterol Hepatol 2000; 12:855-862.
Thomas G A, Rhodes J, Ingram J R. Mechanisms of disease: nicotine—a review of its actions in the context of gastrointestinal disease. Nat Clin Pract Gastroenterol Hepatol 2005; 2:536-544.
Ingram J R, Rhodes J, Evans B K, Thomas G A. Preliminary observations of oral nicotine therapy for inflammatory bowel disease: an open-label phase I-II study of tolerance. Inflamm Bowel Dis 2005; 11:1092-1096.
Gaimarri A, Moretti M, Riganti L, Zanardi A, Clementi F, Gotti C. Regulation of neuronal nicotinic receptor traffic and expression. Brain Res Rev 2007; 55:134-143.
Gallowitsch-Puerta M, Tracey K J. Immunologic role of the cholinergic anti-inflammatory pathway and the nicotinic acetylcholine alpha 7 receptor. Ann N Y Acad Sci 2005; 1062:209-219.
Tracey K J. The inflammatory reflex. Nature 2002; 420:853-859.
Wang H, Yu M, Ochani M, Amella C A, Tanovic M, Susarla S, Li J H, Wang H, Yang H, Ulloa L, Al-Abed Y, Czura C J, Tracey K J. Nicotinic acetylcholine receptor alpha7 subunit is an essential regulator of inflammation. Nature 2003; 421:384-388.
de Jonge W J, van der Zanden E P, The FO, Bijlsma M F, van Westerloo D J, Bennink R J, Berthoud H R, Uematsu S, Akira S, van den Wijngaard R M, Boeckxstaens G E. Stimulation of the vagus nerve attenuates macrophage activation by activating the Jak2-STAT3 signaling pathway. Nat Immunol 2005; 6:844-851.
Floto R A, Smith K G. The vagus nerve, macrophages, and nicotine. Lancet 2003; 361:1069-1070.
Ghia J E, Blennerhassett P, Kumar-Ondiveeran H, Verdu E F, Collins S M. The vagus nerve: a tonic inhibitory influence associated with inflammatory bowel disease in a murine model. Gastroenterology 2006; 131:1122-1130.
Mazurov A, Hauser T, Miller C H. Selective alpha7 nicotinic acetylcholine receptor ligands. Curr Med Chem 2006; 13:1567-1584.
Alex, P, Zacho, N., Nguyen, T., Gonzales, L., Chen, T. E., Conklin, L. S., Centola, M, and Li, X. Distinct cytokine patterns identified from multiplex profiles of experimental colitis. Inflammatory Bowel Disease. 2009; 15(3), 341-352
Alex P, Szodoray P, Knowlton N, Dozmorov I M, Turner M, Frank M B, Arthur R E, Willis L, Flinn D, Hynd R F, Carson C, Kumar A, El-Gabalawy H S, Centola M. Multiplex serum cytokine monitoring as a prognostic tool in rheumatoid arthritis. Clin Exp Rheumatol 2007; 25:584-592.
Alex P M, Frank B, Cadwell C, Dozmorov I, Miner P, Brant S, Centola M, Li X. CXCL8, CCL3, CCL4, IL-10 AND IL-17 are primary mediators of the early differential immune response in active patients with inflammatory bowel disease. 15th United European Gastroenterology Week “UEGW 2007” Paris, France, Oct. 27-31, 2007 2007.
Nakayama T, Hieshima K, Nagakubo D, Sato E, Nakayama M, Kawa K, Yoshie O. Selective induction of Th2-attracting chemokines CCL17 and CCL22 in human B cells by latent membrane protein 1 of Epstein-Barr virus. J Virol 2004; 78:1665-1674.
Burdin N, Peronne C, Banchereau J, Rousset F. Epstein-Barr virus transformation induces B lymphocytes to produce human interleukin 10. J Exp Med 1993; 177:295-304.
Vockerodt M, Pinkert D, Smola-Hess S, Michels A, Ransohoff R M, Tesch H, Kube D. The Epstein-Barr virus oncoprotein latent membrane protein 1 induces expression of the chemokine IP-10: importance of mRNA half-life regulation. Int J Cancer 2005; 114:598-605.
Kuhn R, Lohler J, Rennick D, Raj ewsky K, Muller W. Interleukin-10-deficient mice develop chronic enterocolitis 2. Cell 1993; 75:263-274.
Ribbons K A, Thompson J H, Liu X, Pennline K, Clark D A, Miller M J. Anti-inflammatory properties of interleukin-10 administration in hapten-induced colitis. Eur J Pharmacol 1997; 323:245-254.
Lee D H, Esworthy R S, Chu C, Pfeifer G P, Chu F F. Mutation accumulation in the intestine and colon of mice deficient in two intracellular glutathione peroxidases. Cancer Res. 2006 Oct. 15; 66(20):9845-51.
Porsolt R D, Bertin A, Jalfre M. Behavioral despair in mice: a primary screening test for antidepressants. Arch Int Pharmacodyn Ther 1977; 229:327-336.
Zomkowski A D, Santos A R, Rodrigues A L. Evidence for the involvement of the opioid system in the agmatine antidepressant-like effect in the forced swimming test. Neurosci Lett 2005; 381:279-283.

Although the foregoing subject matter has been described in some detail by way of illustration and example for purposes of clarity of understanding, it will be understood by those skilled in the art that certain changes and modifications can be practiced within the scope of the appended claims.

1. A method for treating Crohn's Disease (CD) in a subject in need of treatment thereof, the method comprising administering a specific antagonist of nicotinic acetylcholine receptor alpha 7 (nAChRα7) to the subject in an amount effective to specifically modulate an activity of nicotinic acetylcholine receptor alpha 7 (nAChRα7) in at least one cell of the subject, wherein the specific antagonist of (nAChRα7) is methyllycaconitine (MLA), derivatives thereof, and pharmaceutically acceptable salts thereof, whereby the specific modulating of the activity of nAChRα7 in the at least one cell treats the CD in the subject.

2.-18. (canceled)
